(19) United States
(12) Patent Application Publication
(10) Pub. No.: US 2020/0030072 A1
(43) Pub. Date: Jan. 30, 2020

(54) SHAPE MEMORY PATCH FOR TISSUE REPAIR

(71) Applicants: Children's Hospital Medical Center, Cincinnati, OH (US); University of Cincinnati, Cincinnati, OH (US)

(72) Inventors: Jose Peiro-Ibanez, Cincinnati, OH (US); Marc Oria, Cincinnati, OH (US); Rigwed Tatu, Cincinnati, OH (US); Chia-Ying Lin, Mason, OH (US)

(21) Appl. No.: 16/337,278

(22) PCT Filed: Oct. 5, 2017
(86) PCT No.: PCT/US2017/055331

§ 371 Date: Mar. 27, 2019

Related U.S. Application Data
(60) Provisional application No. 62/404,325, filed on Oct. 5, 2016.

(51) Int. Cl. A61F 2/00; C08G 63/08; C08L 67/04

(52) U.S. Cl. CPC A61F 2/0063; A61F 2/0077; C08G 63/08; C08L 67/04; A61F 2250/0082; A61F 2210/0014; A61F 2002/0086; C08G 2280/00; C08L 2203/02; C08L 2203/16; A61F 2002/0072

(57) ABSTRACT

A patch of material is configured to be applied to human tissue. The patch includes a film comprising poly(L-lactide) and poly(#-caprolactone). The film is configured to self-deploy between a first position and a second position in response to a temperature. The film is applied to the human tissue when the patch of material is in the second position, in which the film has a planar configuration.

### CROSS-REFERENCE TO RELATED APPLICATIONS

The present application claims priority to U.S. Provisional Patent Application Ser. No. 62/404,325, filed on Oct. 5, 2016, and entitled “SHAPE-MEMORY PATCH FOR PRENATAL SPINA BIFIDA REPAIR BY FETOSCOPIC APPROACH,” the complete disclosure of which is expressly incorporated by reference herein.

### BACKGROUND OF THE PRESENT DISCLOSURE

Defects, injuries, and deformations of the human body may occur in various tissues, muscles, ligaments, organs, and/or other bodily materials. For example, a hernia may be a bulge or swelling in bodily tissues and/or organs which may protrude relative to the outer skin surface. Additionally, various tissues and/or ligaments may become damaged (e.g., torn), resulting in, for example, shoulder rotator cuff injuries and knee injuries. Even before birth, defects may be present in fetal tissue, for example, neural tube defects which may result in spina bifida.

In the case of spina bifida, there may be different occurrences, types, or condition of the defect, such as open spina bifida or myelomeningocele (“MMC”) in which neural tubes of the spinal column fail to close during the embryological period and various neural elements are exposed. Also, cerebrospinal fluid leak caused by MMC in the developing fetus may result in various anomalies, such as hindbrain herniation and brain-stem concerns. Additionally, the exposure of the extruded spinal cord to the amniotic fluid may introduce a risk of partial or complete paralysis of the body parts beneath the spinal aperture.

Neural tube defects may be addressed before birth through the use of open fetal surgery to reduce the risk of the various aforementioned anomalies. However, open fetal surgery may pose a risk to the mother and may increase the risk of maternal-fetal morbidities. Additionally, current surgical procedures may use natural materials (e.g., collagen/ECM-based materials) or inert materials (e.g., silicone, Teflon) which may have poor mechanical strength and/or require a removal surgery. Additionally, current surgical procedures and materials may use a mesh material to promote tissue in-growth, however, the mesh material is porous and may allow amniotic fluid to contact the location of the neural tube defect, thereby exposing the defect to further damage and/or degradation. Also, current surgical procedures are typically time consuming because the material used to repair the defect must be expanded upon release from a trocar or other surgical instrument before applying to fetal skin or tissue.

As such, time-sensitive and minimally-invasive procedures are needed to best address neural tube defects while minimizing risk to the fetus and the mother.

### SUMMARY OF THE PRESENT DISCLOSURE

In one illustrative embodiment of the present disclosure, a method of applying a patch to human tissue comprises providing a patch of material, where the patch of material is configured to self-deploy between a first position and a second position in response to temperature, and the first position is defined when the patch of material is coiled in a cylindrical shape and the second position is defined when the patch of material is in a planar shape. The method also comprises applying the patch of material to the human tissue when the patch of material is in the second position.

In a further illustrative embodiment of the present disclosure, a patch of material configured to be applied to human tissue comprises a film comprising poly(L-lactide) and poly(ε-caprolactone), where the film is configured to move between a first position and a second position in response to a bodily activation temperature.

In yet another illustrative embodiment of the present disclosure, an assembly for repairing an opening in human tissue comprises an insertion instrument configured for use in a medical procedure and a film configured to move between a first position and a second position. The film is in a cylindrical configuration in the first position and in a planar configuration in the second position. The trocar is configured to receive the film in the first position and apply the film in the second configuration to the human tissue.

The above mentioned and other features of the invention, and the manner of attaining them, will become more apparent and the invention itself will be better understood by reference to the following description of embodiments of the invention taken in conjunction with the accompanying drawings.

### BRIEF DESCRIPTION OF THE DRAWINGS

FIG. 1 is a schematic perspective view of the backside of a fetus with a neural tube defect at the base of the spinal column in the form of spina bifida;

FIG. 2 is a schematic side view of the fetus of FIG. 1 in utero during a procedure to repair the neural tube defect;

FIG. 3 is a schematic view of a shape-memory material in the form of a coiled patch configured to be applied to the neural tube defect on the fetus of FIG. 1;

FIG. 4 is a schematic view of an apparatus configured to form a polymer solution of the patch of FIG. 3;

FIG. 5 is a further schematic view of additional equipment configured to form the patch of FIG. 3;

FIG. 6 is a schematic view of the movement between a first or coiled position of the patch and a second or planar position of the patch of FIG. 3;

FIG. 7 is a schematic view of the process of applying shape-memory properties to the patch of FIG. 6;

FIG. 8 is a series of photographs depicting two illustrative examples of the movement of the patch of FIG. 6 between the first and second positions under different conditions;

FIG. 9 is a schematic view of a testing apparatus of analyzing the permeability of the patch of FIG. 3;

FIG. 10 is a series of micrograph images for analyzing the surface roughness of the patch of FIG. 3;

FIG. 11 is a schematic side view of a procedure to apply the patch of FIG. 3 to a fetus with spina bifida in utero;

FIG. 12A is a schematic perspective view of a trocar for endoscopic surgeries configured to apply the patch of FIG. 3 to the fetus in utero;

FIG. 12B is a perspective view of a portion of fetal tissue affected by spina bifida and positioned to receive the patch from the trocar of FIG. 12A;

FIG. 13A is a schematic perspective view of the trocar of FIG. 12A configured to receive the patch in first or coiled position;

FIG. 13B is a perspective view of the portion of fetal tissue affected by spina bifida and positioned to receive the patch from the trocar of FIG. 13A;

FIG. 14A is a schematic perspective view of the trocar of FIG. 12A after applying the patch to the fetal tissue or skin;

FIG. 14B is a perspective view of the portion of fetal tissue on which the patch has been applied;

FIG. 15A is a schematic view of a suture to be applied to the skin after applying the patch of FIG. 14B thereto;

FIG. 15B is a perspective view of the fetal tissue receiving the suture(s) of FIG. 15A and concealing the patch;

FIG. 16 is a perspective view of the fetal tissue after the suture(s) of FIG. 15B have been applied thereto and the patch is concealed thereunder; and

FIG. 17 is a perspective view of the portion of fetal tissue sutured in FIG. 16 with a second layer or patch of the shape-memory material applied thereto and configured to conceal the sutured tissue.

### DETAILED DESCRIPTION OF THE DRAWINGS

Corresponding reference characters indicate corresponding parts throughout the several views. Unless stated otherwise the drawings are proportional.

The embodiments disclosed below are not intended to be exhaustive or to limit the invention to the precise forms disclosed in the following detailed description. Rather, the embodiments are chosen and described so that others skilled in the art may utilize their teachings. While the present disclosure is primarily directed to a shape-memory material and method of application for neural tube defects, such as spina bifida, the shape-memory material and methods disclosed herein may be applicable to other surgical or non-surgical procedures and to all portions of a body, including any fetal tissue, human adult tissue, and other bodily tissues, organs, muscles, tendons, ligaments, or any other bodily materials.

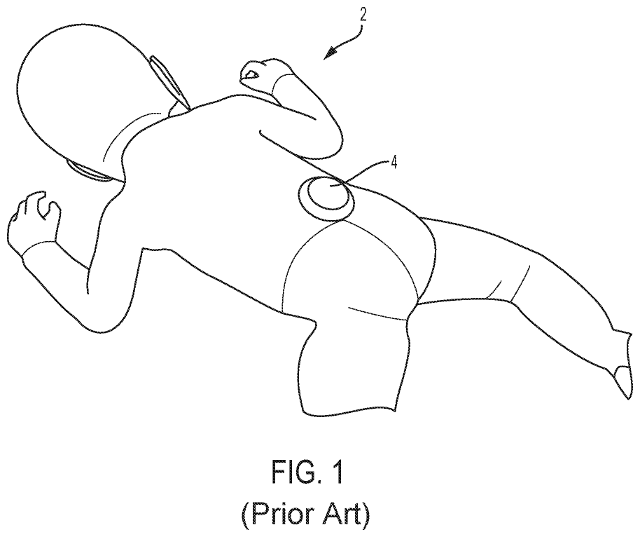

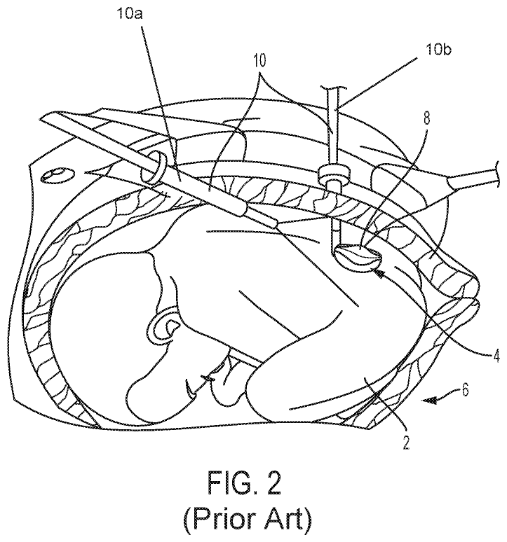

With reference to FIGS. 1 and 2, a defect in human tissue is shown. More particularly, FIGS. 1 and 2 illustratively disclose a human fetus 2 during a fetoscopic surgery within the body of the mother. In particular, fetus 2 has a neural tube defect, such as spina bifida shown at 4, such as open spina bifida or myelomeningocele (“MMC”) in which neural tubes of the spinal column fail to close during the embryological period and various neural elements are exposed. The risk of defect 4 is that exposed portions of the spine may be exposed to and consequently degraded by amniotic fluid (e.g., pH of approximately 7.5-8.0). Additionally, a further risk of defect 4 is that cerebrospinal fluid may leak. Spina bifida and other such defects 4 may result in urinary dysfunction, intestinal dysfunction, and/or partial or complete paralysis.

As shown in FIG. 2, it may be necessary to attempt to repair or mitigate damage at an opening 8 or other anomaly at the location of defect 4 on the human tissue while fetus 2 is within a uterus 6 of a mother carrying fetus 2. While fetus 2 is in utero, insertion instruments 10, for example at least one trocar or cannula 10a and at least one endoscopic grasper 10b, may be used to repair defect 4, for example through a fetoscopic procedure or closure of the tissue/skin at defect 4. While FIGS. 1 and 2 illustrate human fetus 2, the present disclosure is applicable to any type of defect or concern related to any human tissue. For example, the present disclosure is applicable to human children or adults with a rotator cuff injury, knee injury, diaphragmatic hernia, hiatal hernia, abdominal wall hernia, or other types of defects present in an adult or child. Additionally, the present disclosure is applicable to any type of human tissue defect or location requiring a repair through, for example, a laparoscopic procedure, an arthroscopic procedure, and/or an endoscopic procedure.

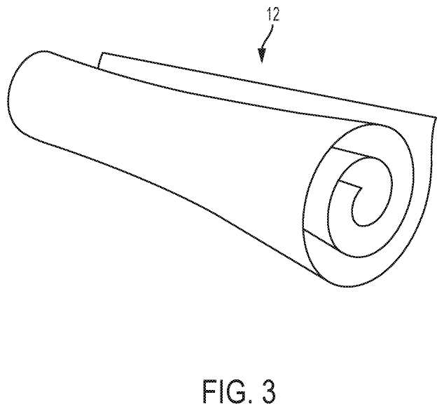

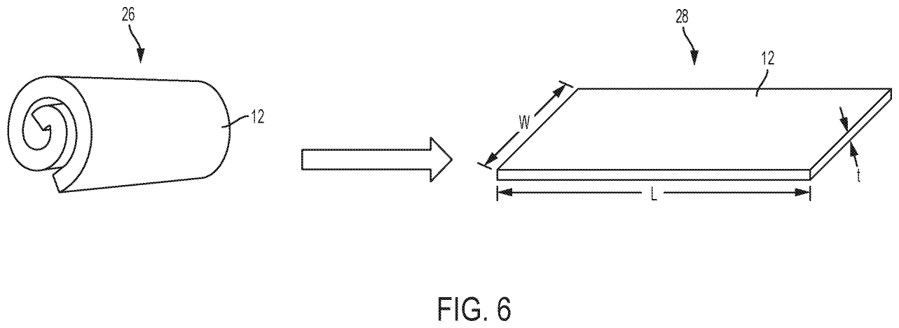

Referring to FIG. 3, a patch or other configuration of material 12 may be used during the surgical procedure of FIG. 2 to repair the defect to the human tissue, even when fetus 2 is in utero. As disclosed herein, patch 12 may be comprised of shape-memory material, thereby allowing patch 12 to automatically move or self-deploy (i.e., without human or other intervention) between a first position 26 (shown in FIG. 3), where patch 12 is coiled into a cylindrical configuration, and a second position 28 (shown in FIG. 6), where patch 12 is expanded or deployed to a planar configuration, in response to a temperature. Patch 12 may then be applied to the human tissue during the surgical procedure of FIG. 2 to conceal and repair defect 4 on the human tissue.

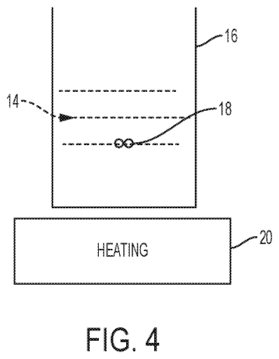

As shown in FIG. 4, patch 12 is initially formed using a polymer solution 14 which may be present in a beaker 16 or other apparatus. More particularly, polymer solution 14 may be a homogenous solution comprised of biodegradable polymers, such as poly(L-lactide) (“PLA”) and poly(ε-caprolactone) (“PCL”). In one embodiment, the weight percentage of PCL within polymer solution 14 may be approximately 10-30% and, more particularly, 13-21%. Additionally, the weight percentage of PLA within polymer solution 14 may be approximately 70-90% and, more particularly, may be approximately 79-87%. Illustratively, polymer solution 14 may contain approximately 17 weight % of PCL and 83 weight % of PLA. To achieve a desired glass-transition temperature of polymer solution 14, the weight percentages of PLA and PCL for patch 12 may be determined using the Flory-Fox equation:

$$\frac{1}{{T}_{g}} = \frac{{w}_{1}}{{T}_{g 1}} + \frac{{w}_{2}}{{T}_{g 2}}$$

where w₁ is the weight fraction of PCL; w₂ is the weight fraction of PLA; Tg₁ is the glass-transition temperature of PCL; Tg₂ is the glass-transition temperature of PLA; and Tg is the glass-transition temperature of polymer solution 14.

By using PCL in polymer solution 14, patch 12 has increased elongation properties, while the use of PLA contributes to an increased tensile modulus of patch 12. The combination of PCL and PLA also contribute to shape-memory properties of a resultant solid material, as disclosed further herein. The elongation and tensile modulus allows patch 12 to be stretch prior to and during a surgical procedure without permanently deforming or otherwise breaking. Additionally, as disclosed herein, the elongation and tensile modulus properties of patch 12 allow patch 12 to self-deploy between first and second positions 26, 28 in response to a temperature.

Additionally, a solvent may be present in polymer solution 14. In one embodiment, the solvent may be chloroform, however, other types of solvents may be used (e.g., dichloromethane). It may be appreciated that the true weights of PLA and PCL are calculated based on the amount of solvent used. In one embodiment, approximately 40 mL of solvent may be used. Polymer solution 14 may be stirred using a conventional stirring device 18, such as a magnetic stirrer. In one embodiment, polymer solution 14 may be stirred with stirring device 18 at a speed of approximately 100-200 rpm.

Beaker 16 and polymer solution 14 may be heated using a heat source 20. Heat source 20 may be heated to approximately 30-40° C. and, more particularly, to 35° C. while polymer solution 14 is being stirred by stirring device 18. Polymer solution 14 may be stirred and heated for approximately 24 hours. Illustrative polymer solution 14 is stirred and heated at approximately 35° C. for approximately 24 hours before being separated into two batches (e.g., 15 mL each, where approximately 10 mL out of 40 mL of solvent may be evaporated during the stirring). The batches may be ultra-sonicated for approximately 10 minutes.

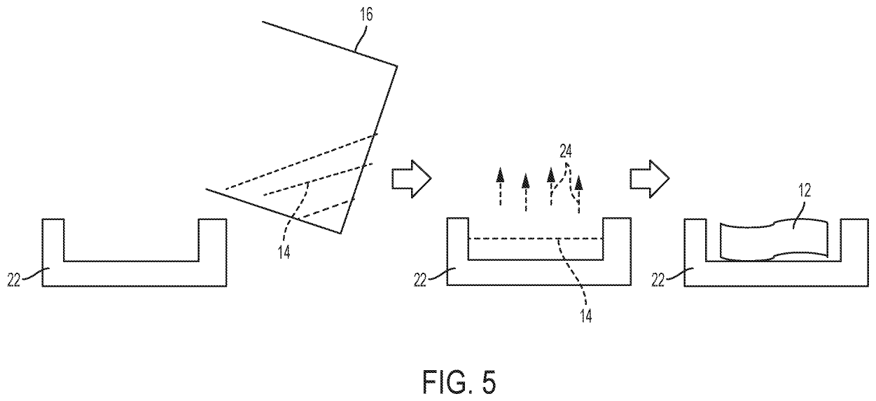

As shown in FIG. 5, once polymer solution 14 is sufficiently stirred and heated for the desired time and at the desired temperature, polymer solution 14 may be poured from beaker 16 into a mold 22. In one embodiment, mold 22 may be comprised of a material with non-sticking properties and the appropriate heat threshold based on the heat of polymer solution 14 entering mold 22. For example, mold 22 may be comprised of polytetrafluoroethylene (“PTFE”).

Once polymer solution is poured into mold 22, polymer solution 14 is cured within mold 22. In one embodiment, a vacuum may be applied to mold 22. The combination of the heat dissipation of polymer solution 14 and the vacuum allows for the solvent (e.g., chloroform) to evaporate from mold 22, as shown by arrows 24. When the solvent has evaporated and polymer solution 14 is sufficiently cured (e.g., as measured by water content, drying time, surface texture, thickness, or any other parameter), polymer solution 14 forms patch 12 in a planar configuration within mold 22. Patch 12 is in solid form and is comprised of PLA and PCL because the solvent has been evaporated therefrom. It may be appreciated that, after evaporation of the solvent, patch 12 does not contain any residual material.

As shown in FIG. 6, patch 12 may have a thickness (t) of approximately 0.05-4.0 mm and, more particularly, 0.5-1.0 mm. In one embodiment, patch 12 may have a thickness of approximately 0.05-0.10 mm and, more particularly, approximately 0.075 mm. Additionally, patch 12 may have a length (L) that varies based on the size of defect 4. Patch 12 also may have a width (W) which may vary based on the size of defect 4. While illustrative patch 12 of FIG. 6 may be configured in a generally rectangular shape as shown, patch 12 may be formed of any size and shape. Mold 22 (FIG. 5) may be sized and shaped to accommodate the varying sizes and shapes of patch 12 (e.g., circle, oval). It may be appreciated that, because insertion instrument 10 has a fixed diameter for receiving patch 12, thickness (t) may vary with the overall shape and size of patch 12. For example, if length (L) and/or width (W) increases, then thickness (t) may be reduced to allow patch 12 to be received within the diameter of insertion instrument 10.

Referring still to FIG. 6, patch 12 is a flexible material configured to be moved to different shapes and configurations. To allow patch 12 to have the desired mechanical properties for movement between different configurations, patch 12 has increased elongation properties and tensile modulus. For example, the Young's Modulus of patch 12 is approximately 2.04±0.40 GPa. Illustrative patch 12 also may have a percent elongation value of approximately 11.3±8.8%. With such properties, patch 12 is configured to self-deploy between a first or un-deployed position 26, defined when patch 12 is coiled into a cylindrical configuration, and a second or deployed position 28, defined when patch 12 in a planar configuration. More particularly, patch 12 is comprised of a shape-memory material in which, in response to a temperature or range of temperatures, patch 12 automatically self-deploys to move between first and second positions 26, 28 without human intervention.

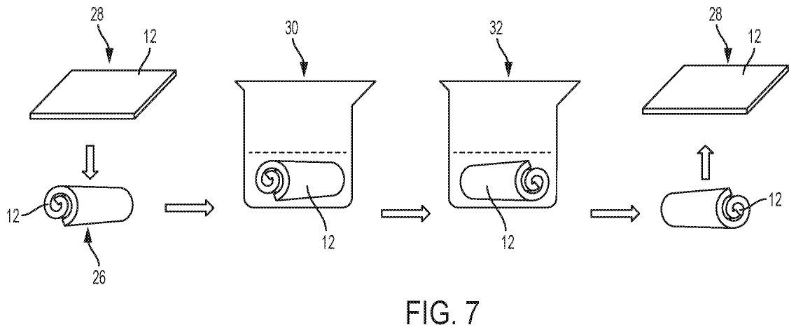

Referring to FIG. 7, in order to apply shape-memory properties to patch 12, patch 12 may initially begin in second position 28 (i.e., the planar configuration). When in second position 28, patch 12 may initially be at room temperature (e.g., approximately 20-22° C.) but patch 12 then may be stretched along length (L) and width (W) (FIG. 6) at a temperature of approximately 25° C.

Referring still to FIG. 7 and the process for applying the shape-memory properties to patch 12, after stretching, patch 12 may then be coiled into first position 26 (i.e., the cylindrical configuration). The movement of patch 12 from second position 28 to first position 26 may occur at approximately 25° C. (i.e., the same temperature at which it was stretched). When in first position 26, heat is applied to patch 12. For example, the temperature of patch 12 may be elevated from 25° C. to a temperature of approximately 45-55° C. and, more particularly, to a temperature of approximately 50° C. In one illustrative embodiment, the activation temperature of patch 12 is generally equal to the glass-transition temperature of patch 12, which is approximately 37.65° C.±1.17° C. In this way, patch 12 may be subjected to temperatures greater than that of the glass-transition temperature thereof. It may be appreciated that patch 12 may be subjected to higher temperatures than 45° C., which may facilitate faster movement between first and second positions 26, 28, as disclosed further herein.

Illustratively, to increase the temperature of patch 12, patch 12 is placed into a fluid bath 30, where the temperature of the fluid is approximately 50° C. Patch 12 is maintained in fluid bath 30 at a temperature of approximately 50° C. for approximately 10 minutes. By placing patch 12 in fluid bath 30, enhanced relaxation of the polymer chains of patch 12 may occur, however, the activation temperature of patch 12 remains at 37° C. More particularly, and as disclosed herein, 37° C. defines the activation temperature for patch 12 because 37° C. defines the glass-transition temperature of polymer solution 14, based on the weight percentages of PCL and PLA. The determination of the activation temperature of patch 12 may be made using thermal test data during differential scanning calorimetry. It may be appreciated that the glass-transition temperature of polymer solution 14, which equates to the activation temperature of patch 12, generally matches the average human body temperature such that patch 12 is configured to self-deploy between first and second positions 26, 28 in response to a human bodily environment.

Referring still to FIG. 7 and the process of applying the shape-memory properties to patch 12, following fluid bath 30, patch 12 is re-coiled in a reverse direction and a temperature thereof is decreased to quench patch 12. For example, patch 12 in the reversed coiled position of first position 26 may be placed in a second fluid bath 32 with fluid at a temperature of approximately 0° C. Patch 12, in the reverse coiled position, may be maintained at 0° C. for approximately 10 minutes.

Referring still to FIG. 7, once quenched, the temperature of patch 12 is increased from approximately 0° C. to the activation temperature of approximately 37° C. For example, to increase the temperature of patch 12 to 37° C., a fluid bath or an oven may be used. Because 37° C. is the activation temperature of patch 12, patch 12 is configured to move between first position 26 to second position 28 at 37° C. automatically and without human or other external intervention due to this shape-memory process. Therefore, after quenching patch 12, the temperature of patch 12 is increased to the activation temperature of approximately 37° C. and patch 12 automatically self-deploys from first position 26 to second position 28. It may be appreciated that patch 12 remains in the reverse-coiled position as when in second fluid bath 32 when increasing the temperature thereof to allow patch 12 to self-deploy from first position 26 to second position 28.

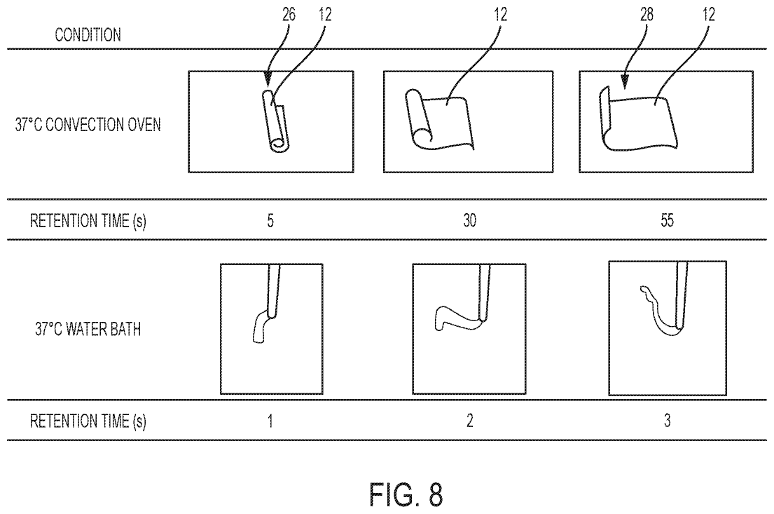

Referring to FIG. 8, to analyze the time required for patch 12 to move between first position 26 and second position 28, patch 12 was tested at 37° C. using air heating and fluid heating. More particularly, the temperature of patch 12 was elevated to 37° C. in a convection oven and the movement between first position 26 and second position 28 lasted approximately 55 seconds. Conversely, when the temperature of patch 12 was increased to 37° C. in a fluid bath (e.g., a water bath), the movement between first position 26 and second position 28 lasted approximately 3-10 seconds and, more particularly, 3 seconds. As such, it may be appreciated that humidity influences the shape-memory and shape-retention properties of patch 12.

Due to the rapid self-deployment or expansion of patch 12, when using patch 12 in an in vivo (e.g., in utero) environment, the movement of patch 12 to second position 28 may occur generally instantaneously, thereby saving time during sensitive surgical procedures and eliminating the need for multiple surgical tools for the deployment of patch 12. Because of the nature of surgical procedures, this rapid self-deployment of patch 12 from first position 26 to second position 28, especially in the presence of a predetermined amount of relative humidity and temperature may be useful.

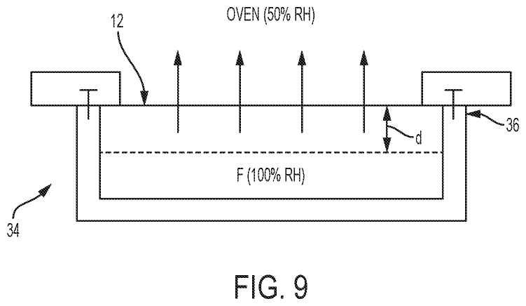

Referring to FIG. 9, patch 12 is configured as a fluid-proof or impermeable material such that patch 12 is generally impermeable to bodily and other fluids. To analyze the permeability properties of patch 12, a water cup test may be performed, which is a commonly-adopted test for water vapor and liquid water transmission of materials, including permeability analysis. Using a test apparatus 34, patch 12 may be sealed to an upper portion of test apparatus 34 at a sealed interface 36 after filling test apparatus 34 with a fluid F at 100% relative humidity (e.g., water). A distance (d) between the surface of fluid F and patch 12 may be approximately 5 mm. Test apparatus 34 with patch 12 sealed thereon may be placed in an oven or other heat source at a temperature of approximately 37° C. and approximately 50% relative humidity. The difference in hydration levels above and below patch 12 creates a pressure gradient in the system, thereby driving water evaporation. Test apparatus 34 with patch 12 sealed thereon may remain at approximately 37° C. for approximately 25 hours and water vapor transmission is recorded. After approximately 25 hours, the weight loss of fluid F was negligible (approximately 0.04 g), thereby demonstrating that patch 12 is generally impermeable to water vapor transmission and, therefore, is applicable for water-tight applications. The impermeability to fluid flow through patch 12 also may be defined by the pore size of patch 12 because, if any pores are present in the material of patch 12, the pores are less than 10 μm, which does not permit fluid flow through patch 12. In this way, patch 12 is applicable for in utero applications because patch 12 is impermeable to the flow of amniotic and other fluids toward the human tissue at the location of defect 4. The impermeability of and water-tight barrier defined by patch 12 may be defined based on the low permeability to liquid water, for example 0.000414 μLcm⁻²min⁻¹H₂O⁻¹.

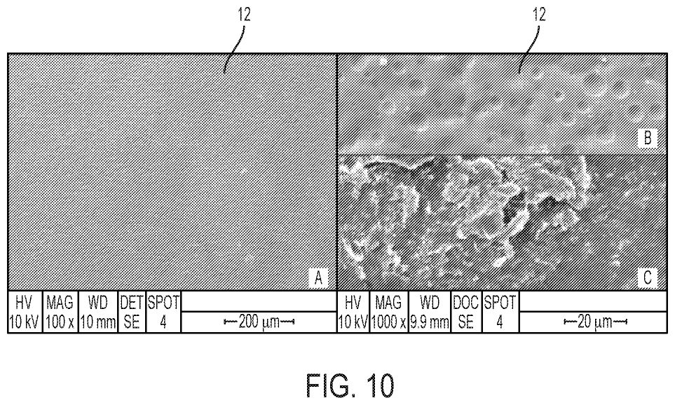

Referring to FIG. 10, the surface roughness of patch 12 indicates that that material of patch 12 is miscible without phase separations. More particularly, as shown in image A of FIG. 10, a scanning electron microscopy image taken at 100× resolution shows that the material of patch 12 has a uniform surface. Additionally, image B of FIG. 10 shows a scanning electron microscopy image taken at 1000× resolution which further discloses that patch 12 has a uniform surface without phase separations. Illustratively, the surface roughness of the material of patch 12 may be approximately 131±8.54 mm. The uniform surface of patch 12 allows for sufficient protein adhesion and cell growth, which, because patch 12 is applied to human tissue, is an important parameter.

Conversely, image C of FIG. 10 shows a scanning electron microscopy image taken at 1000× of a formulation for patch 12 that includes less than 17 weight % of PCL and a glass-transition temperature greater than approximately 40° C. It is clear from image C of FIG. 10 that such formulations result in phase separations, delaminations, and non-uniformity, which may negatively affect the properties of such a patch. Therefore, such formulations that result in the surface roughness of image C in FIG. 10 do not promote sufficient protein adhesion and cell growth, thereby negatively affecting the impact of such a patch on the human tissue.

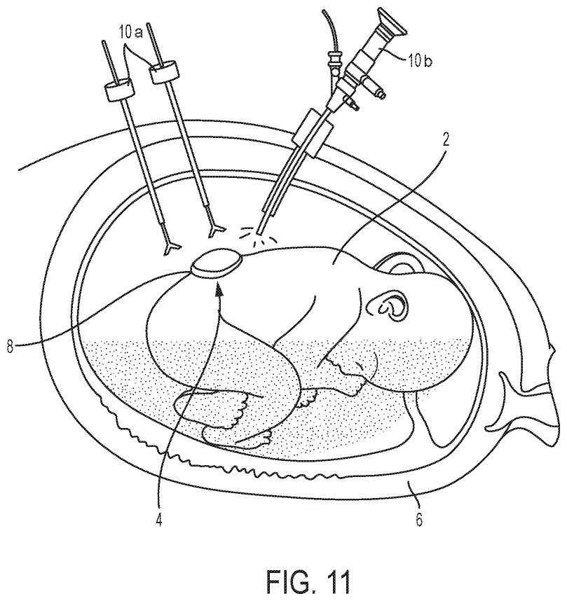

Referring to FIGS. 11-17, in operation, defect 4 is shown, illustratively, as opening 8 at the spine of fetus 2. More particularly, FIG. 11 illustratively discloses that human fetus 2 has a neural tube defect, such as spina bifida, shown at 4. To repair opening 8 or other defects on the human tissue while fetus 2 is within uterus 6 of the mother, insertion instruments 10, for example at least one trocar or cannula, may be used to repair defect 4 during an endoscopic surgical procedure. The mother's abdomen may be surgically opened to expose uterus 6. Additionally, uterus 6 may be surgically opened to expose defect 4. Once defect 4 is exposed, any tissue covering defect 4 and/or at the location of opening 8 may be removed to further expose the affected area on fetus 2.

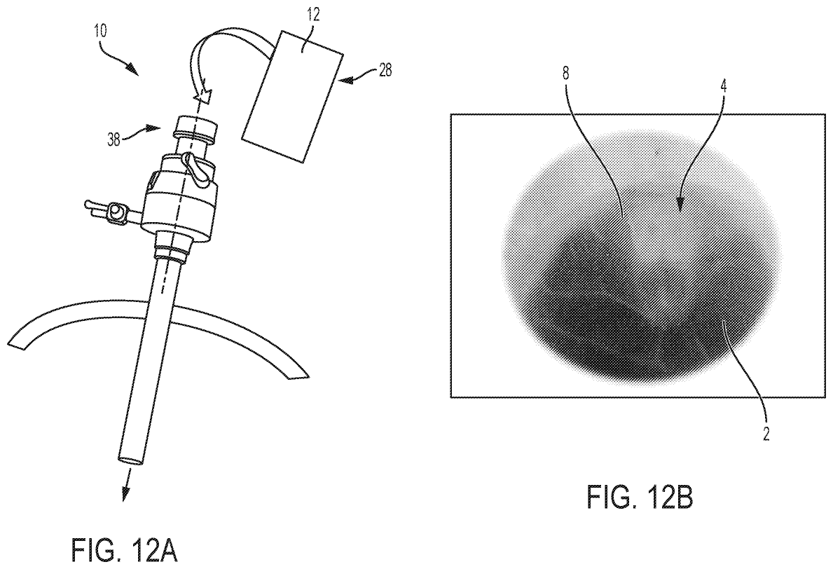

As shown in FIGS. 12A and 12B, with defect 4 exposed, the spinal cord may be freed from any surrounding tissue. The location of defect 4 is then ready to be repaired, as shown in FIG. 12B. Using insertion instrument 10, such as a trocar 10a, in combination with at least one endoscopic grasper 10b, patch 12 may be prepared in the planar configuration of second position 28, as shown in FIG. 12A. Patch 12 is then selected to be inserted into an upper end portion 38 of insertion instrument 10 (e.g., trocar or cannula). In one embodiment, a kit or assembly may be used within insertion instrument(s) 10 to allow the doctor or other medical professional to select the appropriate size and shape of patch 12 for the procedure. The kit may include a plurality of patches 12, with varying shapes and sizes.

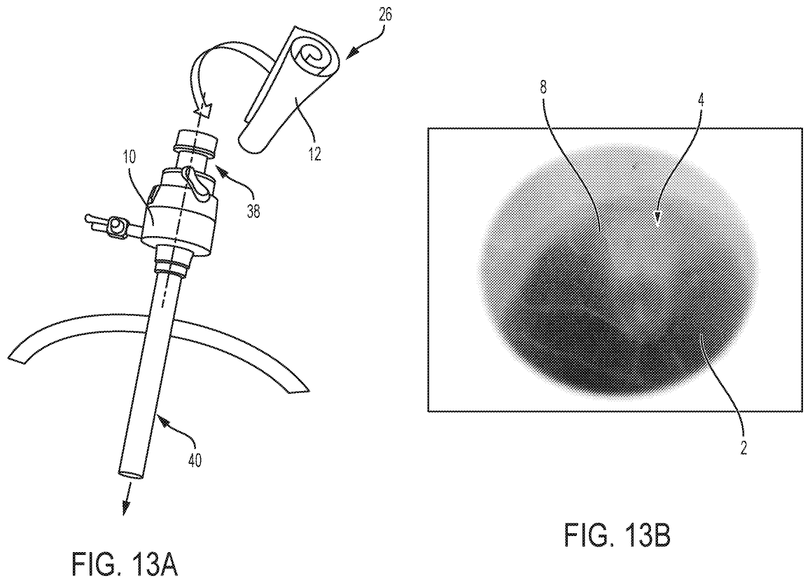

Referring to FIGS. 13A and 13B, before patch 12 is inserted into the bore at upper end portion 38 of insertion instrument 10, patch 12 is moved from second position 28 to first position 26 in order to be inserted therein. More particularly, patch 12 is introduced to insertion instrument 10 in an environment with a temperature below the activation temperature of patch 12. In this way, patch 12 automatically moves or self-deploys to first position 26 from second position 28 prior to entry into upper end portion 38 of insertion instrument 10. Additionally, if patch 12 is already in first position 26 and maintained in an environment below the activation temperature thereof, then patch 12 is maintained in first position 26 in preparation for being received into upper end portion 38. As such, patch 12 is inserted into insertion instrument 10 in the first (i.e., un-deployed) position 26.

When in first position 26, patch 12 is inserted into upper end portion 38 of insertion instrument 10. The cylindrical configuration of patch 12 in first position 26 has a diameter less than a diameter of a bore at upper end portion 38 of insertion instrument 10 and also a diameter less than that of a bore at a lower end portion 40 of insertion instrument 10. For example, the diameter of patch 12 in first position 26 may be less than 3 mm, when a trocar with a 3-mm bore is used, and, more particularly, may be less than 2 mm for other sizes and configurations of trocars or insertion instruments 10. In this way, and as disclosed further herein, while in first position 26, patch 12 is configured to be moved through the longitudinal length of insertion instrument 10 for applying to defect 4 of fetus 2. It may be appreciated that upper end portion 38 is outside of the mother's body, however, lower end portion 40 may be positioned within the mother's body at the location of defect 4 such that patch 12 can be inserted into insertion instrument 10 outside of the body and moved to lower end portion 40 within the body.

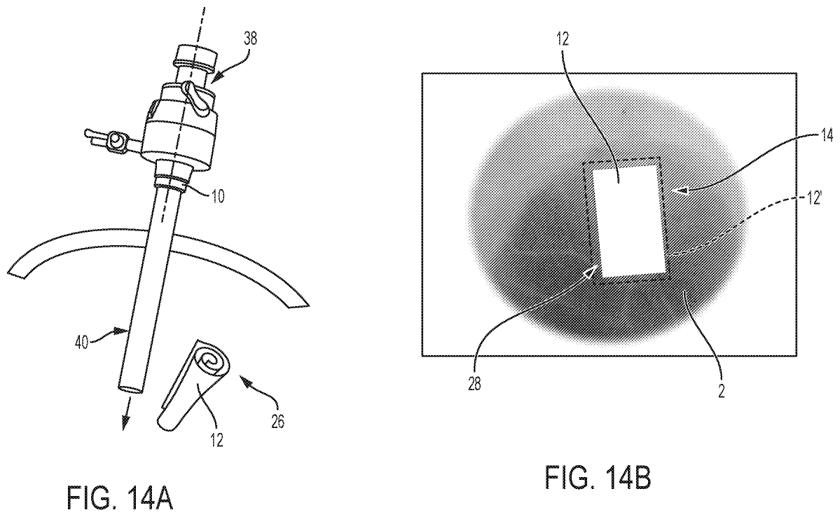

Referring to FIGS. 14A and 14B, patch 12 is moved through insertion instrument 10 and is removed from lower end portion 40 thereof while still in first position 26. Because insertion instrument 10 is positioned at least partially within the mother's body during the surgical procedure, patch 12 is exposed to increased temperature and/or relative humidity and begins to self-expand from first position 26 to second position 28 in response to the body temperature of the mother. For example, as long as the mother's body temperature is at least approximately 37° C., upon exiting insertion instrument 10, patch 12 automatically moves to second position 28 in preparation for applying to the human tissue of fetus 2 at the location of defect 4. More particularly, once patch 12 is intrauterine, patch 12 (in first position 26) may be submerged into amniotic fluid which is at a temperature greater than the activation temperature of patch 12 (i.e., greater than 37° C.). As such, patch 12 automatically deploys to second position 28 within the amniotic fluid as patch 12 is moved to the location of defect 4. Also, due to the fluid environment at which patch 12 self-deploys to second position 28, patch 12 rapidly deploys to second position 28 within seconds, rather than minutes, thereby decreasing the time of the surgical procedure.

As shown in FIG. 14B, patch 12 is applied to defect 4 in second position 28 to conceal defect 4. More particularly, lower end portion 40 of insertion instrument 10 may include forceps or another mechanism for holding patch 12 and applying to defect 4. Alternatively, a second surgical instrument may be used to extract patch 12 from insertion instrument 10 and apply to defect 4. Because patch 12 self-expands in response to the activation temperature, the use of patch 12 during a surgical procedures saves time during sensitive surgeries and eliminates the need for a cumbersome process of patch deployment using various surgical tools.

Using sutures and/or biocompatible adhesive, patch 12 is retained at the location of defect 4. In one embodiment, when patch 12 is joined with the tissue, an additional or second patch 12′, also comprised of the material of patch 12, may be applied over patch 12 and joined with the human tissue using sutures, staples, biocompatible adhesive, and/or any other type of biocompatible coupler. Patch 12′ may be used to conceal patch 12 and/or any biocompatible couplers used to join patch 12 to the fetal tissue. Additionally, and as disclosed herein, because patch 12 is impermeable to fluids, defect 4 is concealed from amniotic and other fluids, which could cause further degradation and/or damage at the location of defect 4. Further, and also as disclosed herein, because patch 12 has a uniform surface without any phase separations, patch 12 promotes protein adhesion and cell growth at the location thereof for further repair to the location of defect 4.

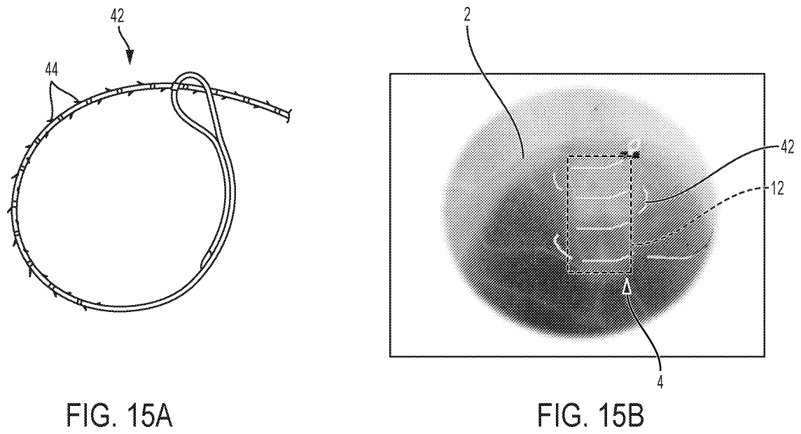

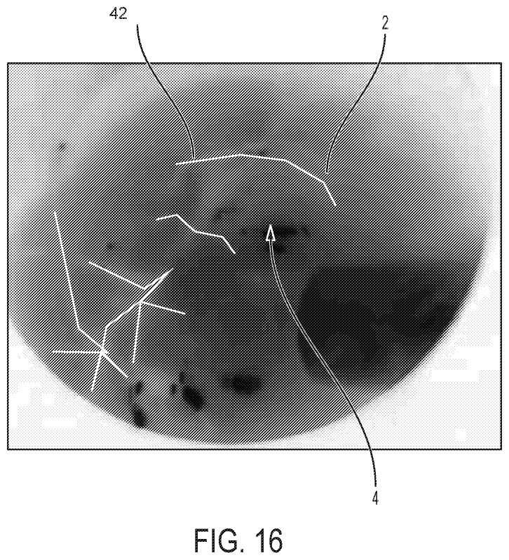

Referring to FIGS. 15A, 15B, and 16, a suture 42 may be used at the location of defect 4 to close the tissue once patch 12 is applied thereto. Suture 42 may include a plurality of projections or barbs 44 to facilitate skin closure. As such, the skin or tissue at the location of defect 4 may be closed around patch 12, thereby defining patch 12 as an inner patch configured to be positioned under at least a portion of skin or tissue.

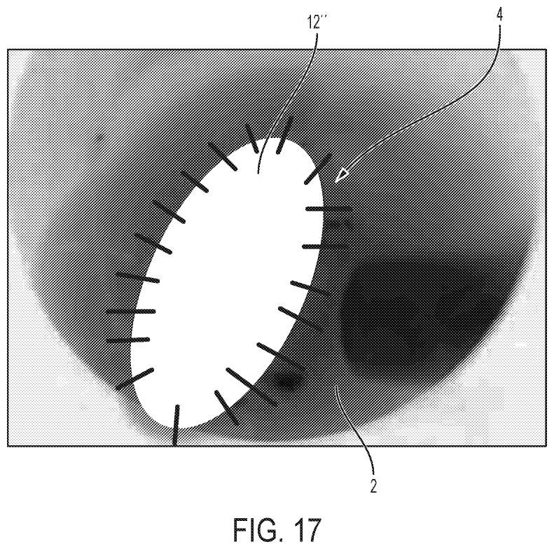

Referring to FIG. 17, a third patch 12″ may be used as a “substitute skin” or outer patch configured to be applied over a portion of skin or tissue and, more particularly, applied over sutures 42. In this way, third patch 12″ may be used to conceal patches 12, 12′ and/or any biocompatible couplers applied thereto. Illustratively, patch 12″ is retained external to the fetal tissue while patches 12, 12′ are retained internally to the fetal skin. Third patch 12″ may be comprised of the same material as patch 12 and, therefore, is impermeable to fluids. When patches 12, 12′, and/or 12″ are applied to the location of defect 4, uterus 6 is then closed using sutures and/or biocompatible adhesive and the mother's abdomen is further closed to conclude the surgical procedure. At this time, the use of at least patch 12 is configured to conceal defect 4 such that defect 4 is not exposed to further degradation or damage due to the presence of amniotic fluid.

It may be appreciated that, because patch 12 is comprised of biodegradable and biocompatible materials, patch 12 may deteriorate over time and is assimilated into the body, thereby eliminating the need for a removal surgery. In this way, defect 4 may be repaired during the surgical procedure disclosed herein but without the need for further surgical procedures, either in utero and/or after fetus 2 is born.

While this invention has been described as having an exemplary design, the present invention may be further modified within the spirit and scope of this disclosure. This application is therefore intended to cover any variations, uses, or adaptations of the invention using its general principles. Further, this application is intended to cover such departures from the present disclosure as come within known or customary practices in the art to which this invention pertains.

What is claimed is:

1. A method of applying a patch to human tissue, comprising:
providing a patch of material, where the patch of material is configured to self-deploy between a first position and a second position in response to temperature; and
applying the patch of material to the human tissue when the patch of material is in the second position.

2. The method of claim 1, further comprising positioning the patch of material into a proximal end of a bore of a surgical instrument when the patch of material is in the first position.

3. The method of claim 2, further comprising removing the patch of material from the bore at a distal end of the surgical instrument when the patch of material is in the second position.

4. The method of claim 3, further comprising self-deploying the patch of material from the first position to the second position following removing the patch of material from the surgical instrument and before applying the patch of material to the human tissue.

5. The method of claim 4, wherein self-deploying the patch of material occurs in response to a body temperature in a range of 27-40° C.

6. The method of claim 1, wherein applying the patch of material to the human tissue includes applying a first patch of material to the human tissue and applying a second patch of material over the first patch of material.

7. The method of claim 1, wherein the first position id defined when the patch of material is coiled in a cylindrical shape and the second position is defined when the patch of material is in a planar shape.

8. The method of claim 7, wherein forming the patch of material includes:
mixing a solution of poly(L-lactide), poly(ε-caprolactone), and a solvent;
curing the solution; and
forming the cured solution into a film.

9. The method of claim 8, wherein the film has a thickness less than 1.0 mm.

10. The method of claim 8, wherein patch of material comprises approximately 70-90 weight percent of poly(L-lactide) and approximately 10-30 weight percent of poly(ε-caprolactone).

11. The method of claim 8, wherein the solvent is chloroform.

12. The method of claim 1, wherein the patch of material is impermeable to fluids and a pore size of the patch of material is less than 10 μm.

13. The method of claim 1, wherein providing a patch of material further comprises:
forming a patch of material in a planar shape;
coiling the patch of material into a cylindrical shape in a first direction;
increasing a temperature of the patch of material to a first temperature when in the cylindrical shape in the first direction;
coiling the patch of material into the cylindrical shape in a second direction;
decreasing the temperature of the patch of material to a second temperature when in the cylindrical shape in the second direction; and
increasing the temperature of the patch of material to the first temperature.

14. The method of claim 1, wherein applying the patch of material to human tissue occurs in utero.

15. The method of claim 14, wherein applying the patch of material includes applying the patch to a fetus with spina bifida.

16. A patch of material configured to be applied to human tissue, comprising:
a film comprising poly(L-lactide) and poly(ε-caprolactone), wherein the film is configured to self-deploy between a first position and a second position in response to a bodily activation temperature.

17. The patch of claim 16, wherein the film has a thickness less than 1.0 mm.

18. The patch of claim 16, wherein the patch is configured to be applied to the human tissue in utero.

19. The patch of claim 16, wherein a pore size of the film is less than 10 μm.

20. The patch of claim 16, wherein the bodily activation temperature of the film is approximately 36-38° C.

21. The patch of claim 16, wherein the film comprises approximately 70-90 weight percent of poly(L-lactide) and approximately 10-30 weight percent of poly(ε-caprolactone).

22. An assembly for repairing an opening in human tissue, comprising:
an insertion instrument configured for use in a medical procedure; and
a film configured to self-deploy between a first position and a second position in response to a bodily activation temperature, the film being in a cylindrical configuration in the first position and in a planar configuration in the second position, and the insertion instrument being configured to receive the film in the first position and apply the film in the second configuration to the human tissue.

23. The assembly of claim 22, wherein the insertion instrument is configured for an in-utero medical procedure and the film is applied to an opening of human tissue adjacent a spine.

24. The assembly of claim 20, wherein the film is configured to self-deploy between the first and second position in response to a temperature of the human tissue in a range of 27-40° C.

25. The assembly of claim 24, wherein the temperature is approximately 36-38° C.

26. The assembly of claim 22, wherein the film comprises poly(L-lactide) and poly(ε-caprolactone).

27. The assembly of claim 22, further comprising a second film configured to self-deploy between the first position and the second position in response to the bodily activation temperature, and the insertion instrument being configured to receive the second film in the first position and apply the second film in the second configuration to the human tissue at a position adjacent the first film.

28. The assembly of claim 22, wherein the insertion instrument defines a trocar configured to receive the film in the first position.
